(19) United States
(12) Patent Application Publication
(10) Pub. No.: US 2025/0073371 A1
(43) Pub. Date: Mar. 6, 2025

(54) PROCESS TO OBTAIN THREE-DIMENSIONAL BIODRESSING, THREE- DIMENSIONAL BIODRESSING OBTAINED AND ITS USE

(71) Applicant: IN SITU TERAPIA CELULAR LTDA., Ribeirão Preto, São Paulo (BR)

(72) Inventors: Carolina Caliari OLIVEIRA, Ribeirão Preto, São Paulo (BR); Adriana Oliveira MANFIOLLI, Ribeirânia, São Paulo (BR)

(21) Appl. No.: 18/293,104

(22) PCT Filed: Jun. 23, 2022
(86) PCT No.: PCT/BR2022/050235

§ 371 Date: Jan. 29, 2024

(30) Foreign Application Priority Data
Jul. 30, 2021 (BR) 1020210151080

(51) Int. Cl. A61L 15/22; A61L 26/00

(52) U.S. Cl. CPC A61L 15/22; A61L 26/0066; A61L 2300/64

(57) ABSTRACT

The present invention refers to a process to obtain a three-dimensional (3D) biodressing comprising the steps of (a) isolating and culturing mesenchymal cells with human AB serum; (B) performing three-dimensional (3D) bioprinting using a biomaterial such as sodium alginate and cells obtained in step “a”; (c) putting at rest the 3D biodressing obtained after bioprinting; (d) covering the 3D biodressing with a 100 mM calcium chloride solution; (e) washing the 3D biodressing; (f) adding Dulbecco's Modified Eagle Medium (DMEM); and (g) keeping the biodressing in an incubator for up to 15 days. Additionally, the present invention refers to the 3D biodressing obtained comprising, preferably 4% sodium alginate and mesenchymal cells from the umbilical cord (MCUs), which has healing, anti-inflammatory, and analgesic properties. Furthermore, the present invention relates the 3D biodressing to treat patients with chronic wounds and severe burns.

### APPLICATION FIELD

The present invention is part of the field of dressings, specifically biodressings, since it refers to a process to obtain a three-dimensional (3D) biodressing comprising an association between mesenchymal cells and biomaterial to treat patients with chronic wounds and severe burns.

### BACKGROUND OF THE INVENTION

Because the skin plays an essential role in the organism, the loss of its integrity seen in complex wounds results in a series of complications.

Healing is a dynamic and complex process composed of three phases: inflammation, proliferation, and tissue remodeling; thus, each phase is regulated by immune cells, cytokines, and specific growth factors; on the other hand, failures in this process can cause wounds that take a long time to heal, the so-called chronic wounds. Underlying pathologies such as diabetes and sickle cell anemia are commonly related to the onset of chronic wounds. These wounds and other severe skin injuries such as those caused by extensive burns are associated with poor prognosis, long-term treatments, and high costs for health systems.

In this context, regenerative medicine through cell therapy has emerged as an alternative treatment for these patients. Among the cells used therapeutically, mesenchymal cells (MSCs) stand out mainly for their immunomodulatory and regenerative properties. However, the cultivation of these cells under Good Manufacture Practices (GMP) conditions, and their administration methods pose some challenges for cell therapy translation.

Thus, the association between optimal cultivation techniques and the use of tissue engineering strategies and three-dimensional (3D) bioprinting allows for obtaining reproducible three-dimensional cell structures, ensuring the maintenance of the therapeutic potential of cells, the safety of the cell therapy process and its delivery in a less invasive way to patients.

In this sense, the present invention proposes a process to obtain a 3D biodressing that uses MSCs derived from the human umbilical cord or from human adipose tissue and biomaterials, such as sodium alginate produced by the technique of three-dimensional bioprinting to treat patients with chronic wounds and severe burns.

Some documents of the technique describe the use of mesenchymal cells and biomaterial to obtain materials for bioprinting, such as a 3D biodressing.

International patent application No. WO 2016/161944 A1, published on Oct. 13, 2016, by SICHUAN REVOTEK CO., LTD., entitled: “Compositions for cell-based three-dimensional printing,” describes a composition of bio solution (“bio-ink”) comprising a plurality of “bioblocks” that can serve as building blocks of cell-based bioprinting, such as mesenchymal cells (MSCs) cultured in fetal bovine serum. In one experiment to obtain the “bioblocks”, a sodium alginate solution is used for the preparation of the core and the exterior of the “bioblock”. The bioblocks are suspended in a 0.1 M calcium chloride solution for maturation. Furthermore, they can be used for the 3D bioprinting of a scaffold placed on the damaged skin.

Differently, the present invention proposes a process for obtaining a 3D biodressing that uses biomaterial (sodium alginate) and mesenchymal cells derived from the human umbilical cord, in a xenoantigen-free culture (in human AB serum) to treat chronic wounds and severe burns. The calcium chloride solution is manipulated after at least 5 minutes to the bioprinting of the biodressing. In the international patent application No. WO 2016/161944 A1, a combination of alginate with collagen, fibrin, hyaluronic acid, and agarose, among others, is used, forming a complex mixture of alginate and other biopolymers, which probably does not produce a bioblock free of xenoantigen. In addition to the bio-block mentioned above being made up of these biomaterials, it is worth mentioning that the cells are deposited on the top and not within the hydrogel, as proposed by the present invention, since it is known in the technique that cell viability decreases if mixed with the biomaterial.

The international patent application No. WO 19122351 A1, published on Jun. 27, 2019, by CELLINK AB and ENGITIX LTD, entitled: “Tissue-specific human bioinks for the physiological 3D-bioprinting of human tissues for in vitro culture and transplantation,” describes a composition for use in 3D bioprinting comprising a polysaccharide hydrogel solution that can be alginate and an extracellular matrix material specific for tissue human or animal (ECM) obtained in tissue decellularized, in which the composition is supplied with cells, preferably human cells, such as mesenchymal cells or derivatives. In addition, the biodressing obtained is proposed for use in wound healing.

In contrast, the present invention proposes a process of obtaining a 3D biodressing, in which the cultivation of cells is performed in human AB serum. Each centimeter square of the biodressing contains 1×10⁵ mesenchymal cells. After bioprinting, the maturation is achieved with a solution of 100 mM calcium chloride only after 5 to 15 minutes to the bioprinting of the biodressing. The present invention does not use decellularized tissue to obtain the matrix for bioprinting. In general, the process proposed by the present invention is simpler and surprisingly showed that cells mixed with alginate survived and maintained their therapeutic effect. Frequently, the literature mentions that cells do not survive well when only alginate is used.

The article entitled “Wound Dressing Model of Human Umbilical Cord Mesenchymal Stem Cells-Alginates Complex Promotes Skin Wound Healing by Paracrine Signaling” by Song Wang et al., published on Dec. 31, 2015, in Stem Cells International, volume 2016, under DOI No. 10.1155/2016/3269267, describes the preparation of a biodressing comprising mesenchymal cells from the human umbilical cord suspended in alginate solution. In one experiment, 1 mL of cells was mixed with 2 mL of a 150 mM solution of sodium alginate and then solidified in a 150 mM solution of sodium chloride for 2 to 3 minutes, in which the cells were cultured in 10% fetal bovine serum. Nothing is mentioned regarding 3D bioprinting.

Inversely, the present invention proposes a process for obtaining a 3D biodressing, in which cells are cultivated in human AB serum. Each square centimeter of the biodressing contains 1×10⁵ mesenchymal cells. After bioprinting, the maturation with 100 mM calcium chloride solution is performed only after a pause of 10 to 15 minutes for the biodressing bioprinting. The article mentioned above does not use AB serum culture nor mentions the bioprinting process of a biodressing.

The doctoral thesis entitled: “3D Biofabrication of Cell-laden Alginate Hydrogel Structures” published in June 2017 by Atabak Ghanizadeh Tabriz describes a 3D bioprinting method to produce more complex alginate hydrogel structures. Inversely, the present invention proposes a process for obtaining a 3D biodressing in the form of a layered mesh, in which each square centimeter of the biodressing contains 1×10⁵ mesenchymal cells. After bioprinting, maturation is performed with a calcium chloride solution at 100 mM only after a 10 to 15-minute break to bioprint the biodressing.

The master's dissertation entitled: “Development of methodology for the production of human AB serum for supplementation of culture medium intended for the cultivation of mesenchymal cells,” published on Apr. 13, 2017, by Vanessa Tieko Marques dos Santos, only describes the replacement of FBS by human AB serum obtained from human plasma to the culture of mesenchymal cells and nothing is described on the use of these cells in a 3D biodressing with healing, anti-inflammatory, and analgesic properties.

Therefore, unlike the technique, the present invention refers to the preparation of a 3D biodressing using biomaterial, such as sodium alginate, and human umbilical cord mesenchymal cells (MCU) or human adipose tissue, xenoantigen-free cultured (in human AB serum), intended to treat chronic wounds and severe burns.

No prior document describes a xenoantigen-free 3D biodressing, as proposed by the present invention.

Also, an existing technical problem in the process of 3D biodressing is the fact that the biomaterial (sodium alginate) easily undergoes deformation when used for 3D bioprinting. This happens because at low concentrations, its viscosity is low, and the maturation and oxidation process is not enough to maintain its structure; on the contrary, usually, when sodium alginate concentrations are higher, the microenvironment is not suitable to keep the cells alive. In this case, there must be a balance between optimal structure and cell viability.

To solve this technical problem, the present invention proposes a process that prevents the deformation of the biomaterial using approximately 4% concentration of sodium alginate in the optimal structure, obtained by bioprinting and with a more prolonged maturation process with an interval of 10 minutes before adding calcium chloride.

In this sense, the technical effect that prevents the deformation of the alginate is found precisely in the step of adding the calcium chloride solution after at least 5 minutes to the biodressing printing. Thus, this step is essential to maintain the shape of the proposed 3D biodressing.

Therefore, no prior document describes or suggests a process for obtaining a 3D biodressing that uses MSCs from the human umbilical cord or adipose tissue grown in a culture medium supplemented with human AB serum and biomaterial, such as sodium alginate, produced by the three-dimensional bioprinting technique to treat patients with chronic wounds and severe burns.

### SUMMARY OF THE INVENTION

The present invention will provide advantages regarding the process of obtaining three-dimensional biodressing, and the 3D biodressing obtained, enabling an increase in its performance and presenting a more favorable cost/benefit ratio.

In the first aspect, the present invention to obtain a three-dimensional (3D) biodressing comprises the steps of (a) isolating and culturing mesenchymal cells with human AB serum; (b) performing three-dimensional (3D) bioprinting using a biomaterial, such as sodium alginate, and cells obtained in step “a”; (c) putting the obtained 3D biodressing to rest after bioprinting; (d) coating the 3D biodressing with a 100 mM calcium chloride solution; (e) washing the 3D biodressing; (f) adding Dulbecco's modified Eagle's medium (DMEM); and (g) keeping the biodressing in an incubator for up to 15 days in culture medium supplemented with human AB serum.

In a second aspect, the present invention is a 3D biodressing comprising, preferably, 4% sodium alginate, mesenchymal cells derived from the human umbilical cord (MCU), with healing, anti-inflammatory, and analgesic properties.

In a third aspect, the present invention relates the use of 3D biodressing to treat patients with chronic wounds and severe burns.

### BRIEF DESCRIPTION OF FIGURES

The structure and operation of the present invention and its additional advantages can be better understood by referring to the attached figures and the following description.

FIGS. 1A-B refer to a schematic drawing of the bioprinted mesh from a gcode file, where “A” is to the top view of the biodressing, and “B” is the layered distribution of the biodressing.

FIG. 2 is a photograph of the initial appearance of the 3D biodressing formed after bioprinting and maturation in 100 mM calcium chloride solution.

FIG. 3 graphically depicts lymphocyte proliferation assays in the presence of different concentrations of lymphocytes co-cultured with the MCU AB-containing biodressings.

FIGS. 4A-B show the Genesis 3DBS bioprinter used, where “A” refers to the positioning of the 3D bioprinter in the laminar flow cabinet, and “B” refers to the detail of the extruder nozzles of the bioprinter.

FIG. 5 graphically represents the kinetics of cell viability of bioprinted FBS MCUs.

FIG. 6 graphically represents the kinetics of cell viability of bioprinted AB MCUS.

FIG. 7 graphically represents the kinetics of cell viability of bioprinted MCUs in the presence of DMEM culture medium supplemented with 10% FBS and DMEM culture medium supplemented with 10% (PRP) human platelet-rich plasma.

FIG. 8 graphically represents the comparison of cell viability of MCUs in the biodressing in culture medium with 10% PRP and 10% human AB serum.

FIG. 9A-D represents confocal microscopy images of the 3D biodressings produced on the Genesis-3DBS bioprinter. A-D are CFSE-stained mesenchymal cells at 1, 5, 7, and 10 days after bioprinting.

FIG. 10A-L represents images of a histological evaluation of epidermal re-epithelialization in human skin fragments incubated with the evaluated product 3D biodressing, where “A-C” refers to the histological section of ex vivo skin submitted to scalpel injury followed by placebo treatment, “D-F” refers to the histological section of ex vivo skin submitted to scalpel injury followed by treatment with the 3D biodressing, “G-I” refers to the histological section of ex vivo skin submitted to punch injury followed by placebo treatment, “J-L” refers to the histological section of ex vivo skin submitted to punch injury followed by a treatment with the 3D biodressing, in which the bar reference corresponds to 50 μm.

FIG. 11 graphically represents the effect of the evaluated 3D biodressing product on the production of TGF β in human skin culture subjected to tissue injury with a scalpel and punch. Data are expressed as the mean±standard deviation of 4 replicates (ANOVA-Bonferroni).

FIG. 12 graphically represents the effect of the evaluated 3D biodressing product on the production of KGF in human skin culture subjected to tissue injury with a scalpel and punch. Data are expressed as the mean±standard deviation of 4 replicates (ANOVA-Bonferroni).

FIG. 13 graphically represents the lymphocyte proliferation inhibition potential of 3D biodressings.

FIG. 14 graphically represents the lymphocyte proliferation inhibition potential of 3D biodressings.

FIG. 15A-B graphically depicts the effect of biodressing supernatant with MSCs collected 3 (A) and 7 (B) days after biodressing printing on carrageenan-induced hyperalgesia in rats.

FIG. 16 graphically represents the effect of MSCs biodressing supernatant collected 3 and 7 days after biodressing printing on carrageenan-induced hyperalgesia in rats.

### A DETAILED DESCRIPTION OF THE INVENTION

Although the present invention may be susceptible to different implementations, the design and the detailed discussion show a preferred implementation with the understanding that the present invention should be considered an exemplification of the principles of the invention and is not intended to be limited to what has been described in this report.

The present invention refers to a process to obtain a 3D biodressing comprising the following steps:
a) Isolate and cultivate mesenchymal cells with 5 to 15% human AB serum;
b) Perform three-dimensional (3D) bioprinting with 1 to 6% of a biomaterial and 0.5×10⁵ to 1×10⁶ of cells obtained in step “a”;
c) Rest the 3D biodressing obtained for at least 5 minutes after bioprinting;
d) Cover the 3D biodressing with a 100 mM calcium chloride solution for 5 to 15 minutes;
e) Wash the 3D biodressing at least 3 times in a saline solution;
f) Add Dulbecco's-modified Eagle's culture medium (DMEM) comprising 5 to 15% fetal bovine serum or human AB serum; and
g) Keep it in an incubator at 33 to 37° C. and 5 to 7% CO₂ for 1 to 15 days.

In step “a,” the mesenchymal cells are selected among human adipose tissue-derived mesenchymal cells and human umbilical cord-derived mesenchymal cells.

In a preferred implementation, mesenchymal cells derived from MCUs are used.

The isolation and cultivation of these cells are carried out according to widely disseminated state-of-the-art techniques. However, it is worth noting that human AB serum is used so that the whole process is performed under xenoantigen-free conditions. Alternatively, fetal bovine serum (FBS) can be used in addition human platelet-rich plasma (PRP).

After the isolation and cultivation of mesenchymal cells, step “b” of three-dimensional bioprinting is performed.

From the 3D bioprinter used, compatible files containing the source code are obtained to bioprint the biodressing mesh, its size, and 2 to 10 layers to generate three-dimensional structures of 1 cm² to 100 cm² using an image slicing software.

Such file for the 3D biodressing must consist of the layer-by-layer arrangement of 2 to 10 layers of a scheme, where the cells are arranged equidistantly. Preferably, the 3D biodressing has 2 to 10 layers, more preferably 3 layers. The pores, in turn, allow the passage of gases and nutrients to maintain cell viability.

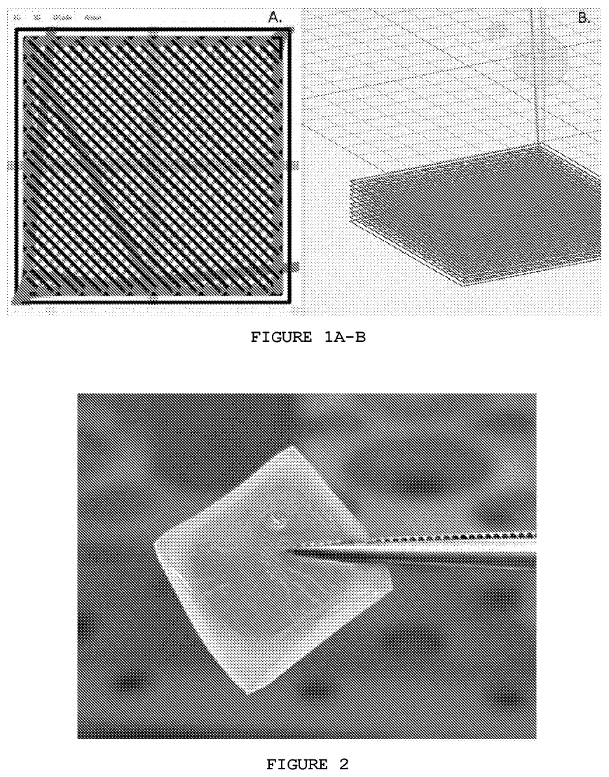

FIG. 1A-B represents a schematic design of the bioprinted mesh from a gcode file, where A is the top view of the dressing and B is the layered distribution of the biodressing.

For extrusion-based bioprinting, a single syringe system is used to inject viscous solutions (bioinks). The syringe is loaded with a biomaterial.

The biomaterial is selected from sodium alginate, collagen, hyaluronic acid, gelatin or cellulose.

In one implementation of the invention, the biomaterial is 4% sodium alginate w/v.

Thus, the structure of the biodressing is generated and printed on sterile culture plates from a model created in CAD software.

Also, in step “b”, to bioprint the cells, after asepsis, the bioprinter is installed inside a laminar flow cabin, which guarantees the sterility of the bioprinted product. Cells are trypsinized, counted, and resuspended in 1 to 6% biomaterial at a concentration of 0.5×10⁵ to 1.0×10⁶ cells/mL, preferably 0.4×10⁶ cells/mL. For this, the extruder nozzle of the bioprinter is filled with the cells in suspension.

Thus, each square centimeter of biodressing comprises 0.1×10⁶ cells in 250 μL of biomaterial.

After bioprinting, in step “C”, the 3D biodressing remains at rest for at least 5 minutes; preferably, for 10 to 15 minutes. Alternatively, the 3D biodressing is left at rest for 30 minutes.

This pause is essential to obtain the 3D biodressing, as it helps maintain the structure of the biodressing before adding calcium chloride.

Shortly after the pause, in step “d”, the 3D biodressing is covered with 100 mM calcium chloride (CaCl₂)) solution for 5 to 20 minutes, preferably 10 minutes, to induce post-print crosslinking.

In step “e”, the biodressing is washed at least 3 times in a saline solution, which is selected from the group consisting of phosphate-buffered saline (PBS), saline or culture medium, in which the saline solution is preferably PBS.

After washing, in step “f”, the DMEM culture medium containing 5 to 15% fetal bovine serum (FBS) or human AB serum is added.

Alternatively, 10% platelet-rich plasma (PRP) is added to replace human FBS or AB sera after bioprinting the cells to enhance their growth in the 3D biodressing from the first day of culture.

Finally, in step “g”, the biodressing is kept in an incubator at 35 to 37° C. and 5 to 7% CO₂ for 1 to 15 days, preferably at 37° C. and 5% CO₂, without changing the culture medium.

During the period above of 1 to 15 days, the biodressing obtained is already ready for use and can be used on any day within that period. However, ideally, it should be used after 3 (three) days of incubation in a CO₂ incubator because the cells reach a proliferation peak on the third day.

For the application, the biodressing must be removed from the culture plate with sterile forceps, washed in saline solution, and applied to the wound/burn.

FIG. 2 is a photograph of the 3D biodressing obtained according to the process of the present invention.

Therefore, surprisingly, the process described here makes it possible to obtain a 3D biodressing composed MCU cells cultured free of xenoantigen-(MCU AB) that shows suitable cell viability after bioprinting for up to 15 days after bioprinting.

Thus, the present invention also refers to the 3D biodressing obtained according to the process described herein.

The 3D biodressing comprises:
from 0.5×10⁵ to 1×10⁶% of mesenchymal cells derived from human adipose tissue (ADSCs) and cells derived from the human umbilical cord (MCUs); and
from 1 to 6% of biomaterial.

In a preferred implementation, the 3D biodressing comprises mesenchymal cells derived the MCUs.

The biomaterial is selected from sodium alginate, collagen, hyaluronic acid, gelatin or cellulose.

In a preferred implementation, the biomaterial is 4% sodium alginate w/v.

The 3D biodressing comprises at least two layers, preferably 2 to 10, in which each square centimeter of biodressing shall consist of 0.1×10⁶ cells in 250 μL of biomaterial.

The size of the biodressing can vary between 1 to 100 cm².

The 3D biodressing has cell viability of up to 15 days after bioprinting. For this purpose, in vitro assays of resazurin viability and confocal microscopy were performed.

Also, biodressing has healing, anti-inflammatory, and analgesic properties.

Therefore, the 3D biodressing of the present invention is obtained by three-dimensional bioprinting according to the process described herein.

The present invention proposes using 3D biodressing for wound healing of different etiologies.

The healing, anti-inflammatory, and analgesic properties of the 3D biodressing of the present invention favor its use as an alternative treatment for patients with chronic wounds and severe burns, such as 2 to 4-degree burns and extensive burns on body surfaces above 10%, improving their prognosis and quality of life and minimizing the burden that the treatment represents to health systems.

Additionally, it is worth emphasizing that the ex vitro healing assay on human skin showed that the biodressings accelerated and improved the healing process and increased the production of growth factors TGF-β and KGF, which are essential during the healing process.

Regarding the immunomodulatory potential of the biodressings of the present invention, the in vitro lymphocyte immunosuppression assay demonstrated that 3D biodressings decreased the proliferation of T lymphocytes. Also, the analgesic effect of the 3D biodressing was evaluated in an experimental model of carrageenan-induced hyperalgesia in rats.

Therefore, the results obtained by various tests will be presented below. They are promising since this product complies with characteristics recommended by the current regulations (FDA and Anvisa), with superiority.

### Preferred Accomplishments of the Invention

For reference purposes, without limiting the possibilities of processes of isolation and cultivation of mesenchymal cells, the following are examples of processes of isolation and cultivation of these cells.

Isolation and Cultivation of Mesenchymal Cells Derived from human adipose tissue (ADSCs):

Mesenchymal cells are obtained from human adipose tissue from liposuction surgeries. Adipose tissue is kept in PBS with 10% antibiotic and antifungal solution (Gibco) at 4-8° C. for 6 hours. The process of isolating MSCs from adipose tissue consists of fragmentation and subsequent enzymatic digestion of the tissue in a PBS solution containing 30% collagenase type 1 (Gibco). The material is incubated in this solution for 1 hour in a water bath at 37° C. After incubation, collagenase is inactivated by adding an equal volume of culture medium DMEM/F12 (Gibco) 10% FBS (Thermo Scientific). This solution is centrifuged for 10 minutes at 500 g, and the pellet is resuspended in PBS. After centrifugation, the supernatant is discarded and the pellet resuspended in DMEM/F12 10% FBS culture medium. The cell suspension is plated in culture bottles and kept in an oven with 5% CO₂ at 37° C.

After 24-36 hours in culture, all supernatant containing non-adherent cells is removed, and 15% FBS DMEM culture medium is added again; the bottles are kept in an oven (5% CO₂ at 37° C.).

### Isolation, Cultivation, and Expansion of Human Umbilical Cord-Derived Mesenchymal Cells (MCUs):

The entire umbilical cord is processed. Briefly, the umbilical cord is fragmented into pieces of the smallest possible size and submitted to the culture technique using explants that are transferred to culture bottles and cultivated at 37° C. in DMEM medium supplemented with 10% human AB serum, plus L-glutamine, antibiotics (penicillin and streptomycin), and amphotericin B.

Non-adherent cells are removed after 4 to 6 days of cultivation, and adherent cells are cultured under the same conditions until the average confluence of 80% when they are collected by trypsinization. The samples that showed alterations in the serological screening tests are discarded.

After the MCU-characterization, they are used in the bioprinting process of the biodressings. The following is an implementation of the process of the present invention.

### Three-Dimensional Bioprinting:

For three-dimensional bioprinting (additive manufacturing), the 3D bioprinter produced by 3D Biotechnology Solutions (3DBS) was used.

Files containing the parameters to generate three-dimensional structures of 1 cm² or 8 cm² were developed with the Cura software for slicing the image created in Autocad. Files in STL. format and gcode can be recognized by the public domain software Pronterface, compatible with the prototype of bioprinter used.

A single syringe system was used to inject viscous solutions (bioinks). The syringe was loaded with 4% sodium alginate w/v (product number: W201502, Sigma-Aldrich) (HE et al., 2016). The structures of biodressings were generated and printed on plates of sterile cultures from a model created in CAD.

For cell bioprinting, the equipment was installed inside the laminar flow cabin, which guaranteed the sterility of the bioprinted product.

FIG. 4A-B shows the bioprinter Genesis 3DBS used, where “A” refers to the positioning of the 3D bioprinter in the laminar flow chamber and “B” refers to the detail of the extruder nozzles of the bioprinter.

Cells were trypsinized, counted, and resuspended in 4% alginate hydrogel (Sigma-Aldrich) at a concentration of 0.4×10⁶ cells/mL. Thus, each square centimeter of biodressing contained 0.1×10⁶ cells in 250 μL of alginate.

After bioprinting, the 3D biodressings were rested for 10-15 min and covered with 100 mM calcium chloride solution (Sigma-Aldrich). After 10 minutes, this solution was removed, and the biodressings were washed three times in PBS.

Finally, a DMEM culture medium (Gibco) containing 10% fetal bovine serum (Thermo Scientific) or 10% human AB serum was added. The biodressings were kept in an incubator at 37° C., 5% CO₂, without any culture medium exchange.

As an alternative, the present process uses platelet-rich plasma (PRP) as a stimulus for cellular maintenance of the 3D biodressing, which was obtained as follows:

### Platelet-Rich Plasma (PRP) as a Stimulus for Cell Maintenance:

Peripheral venous blood was obtained from healthy volunteer donors aged 26-28 from forearm venoclysis. Blood was collected in tubes containing 3.2% sodium citrate anticoagulant (4.5 mL) (BD, New Jersey, USA).

Platelet-Rich Plasma (PRP) was obtained by centrifuging samples at 200 g for 10 minutes for plasma separation. Subsequently, the supernatant plasma fraction was collected and transferred to a 15 mL falcon tube. Plasma was centrifuged again at 200 g for 10 minutes for platelet concentration. After the second centrifugation, the liquid fraction equivalent to the upper ⅔ of the volume contained in the tube was discarded. The lower ⅓ of the volume was defined as the fraction corresponding to the PRP.

Human AB Serum Rather than Fetal Bovine Serum (FBS):

Human AB serum was used after the standardization of biodressings using conventional culture, where fetal bovine serum (is the primary nutrient of the culture medium, aiming at a culture free of xenoantigens. Human serum was obtained from the processing of common AB plasma after “quarantine” by adding 0.1M CaCl₂ in a 9:1 ratio or 0.01M C₁₂H₂₂CaO₁₄. Tests:

Tests to assess cell identity were performed between the 5th and 6th passage of cells. They consisted of a) tests to assess identity (immunophenotyping by flow cytometry) and b) tests to assess cell potency (multipotential differentiation).

### Tests for the Characterization of Mesenchymal Cells

The cell identity assessment was made by monitoring cell morphology, potential for differentiation into adipocytes and osteocytes and by analyzing the expression profile of surface markers by flow cytometry. These tests define the cells as mesenchymal.

In this sense, human umbilical cord-derived mesenchymal cells were isolated and cultured both under usual conditions (MCU FBS) and under xenoantigen-free conditions (MCU AB). For the cultivation under xenoantigen-free conditions, 10% human AB serum was added to the culture medium to replace fetal bovine serum (FBS). Before use, these cells were characterized and demonstrated to have an identity compatible with mesenchymal cells.

### a) Evaluation of Morphology by Inverted Microscopy.

It was performed at each cell passage.

b) Immunophenotyping by Flow Cytometry, with the Following Markers: CD14, CD31, CD34, CD44, CD45, CD90, CD73, CD105, CD146, CD166, HLA-ABC, HLA-DR, KDR.

To analyze the expression profile of surface markers by flow cytometry, cells were transferred to test tubes and stained directly or indirectly by monoclonal antibodies or isotype controls conjugated to fluorochromes. Various antigens were stained to analyze cell subpopulations. Moreover, the cells were stained with annexin and 7AAD for simultaneous assessment of cell viability. Next, the cells were acquired on the FACSCalibur flow cytometer (Becton Dickinson, BD) and analyzed using the Cellquest (BD) software.

Table 1 demonstrates that the AB MCUs showed a characteristic immunophenotype of mesenchymal cells (HORWITZ et al., 2005) with high expression of the markers: CD73, CD44, CD13, CD29, CD90, CD49, CD54, CD105, CD146, and CD166 and absence or low expression of markers: CD14, CD45, CD106, CD34, CD31, CD338, HLA-DR, and HLA-1.

TABLE 1
Immunophenotypic characterization
of MCU grown under GMP conditions

| Marker | Percentage |
|---|---|
| CD73 | 98.44 |
| CD44 | 97.98 |
| CD13 | 99.25 |
| CD29 | 95.68 |
| CD90 | 99.74 |
| CD49 | 99.07 |
| CD54 | 95.58 |
| CD105 | 98.25 |
| CD146 | 88.05 |
| CD166 | 97.6 |
| CD14 | 1.03 |
| CD45 | 0.41 |
| CD106 | 1.72 |
| CD34 | 0.90 |
| CD31 | 0.51 |
| CD338 | 5.08 |
| HLA-DR | 1.12 |
| HLA-1 | 54.06 |

It is worth mentioning the low expression of HLA-DR and HLA-1, molecules of the major histocompatibility complex responsible for recognizing alloantigens, rejection, and complications in unrelated transplants (DELGADO LA, 2018). This low expression of HLA, compatible with MSCs from different sources (LE BLANC et al., 2003), is a crucial issue in the proposal to produce 3D biodressings from allogeneic sources, demonstrating the feasibility of using pre-established cell banks.

The 3D biodressings composed of allogeneic cells can be stored and made available when necessary, which will represent an advance in treating patients with significant burns since immediate care is required (ATIYEH; GUNN; HAYEK, 2005), and the cultivation of autologous cells is a relatively time-consuming process.

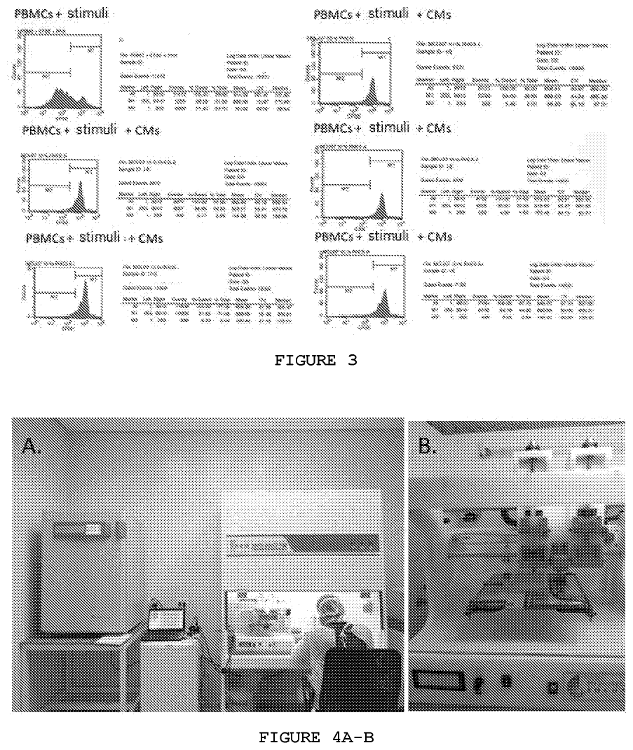

Since MSCs have immunomodulatory properties, the lymphocyte proliferation inhibition assay is a widespread method to evaluate the therapeutic properties of MSCs in culture. In this assay, MSCs are cultured with PBMCs (peripheral blood mononuclear cells) subjected to antigenic stimuli, and the suppression potential to lymphocyte proliferation is evaluated. In this context, AB MCUs, when co-cultured with stimulated lymphocytes, decreased their proliferation from approximately 718 to 5%, proving their immunosuppressive potential in vitro (FIG. 3).

c) Cell Differentiation into Adipocytes and Osteocytes.

1 mL aliquots containing 20,000 cells/mL (differentiation into adipocytes and osteocytes) and 5,000 cells/mL (control) were grown in 24-well plates with coverslips for morphological studies. The medium to induce differentiation into adipocytes was DMEM 15% FBS, supplemented with 10 μM dexamethasone, 10 μg/mL insulin, and 100 mM indomethacin. For osteocytes, 7.5% PBS DMEM was supplemented with 0.10 μM dexamethasone, 100 μM ascorbic acid, and 10 mM β-glycerolphosphate.

### Cell Viability Assays by Confocal Microscopy and Resazurin:

For the cell viability assay by confocal microscopy, the MSCs used in the biodressings were previously stained with the fluorescent cell staining dye carboxyfluorescein ester succinimidyl (CFSE-Thermo Scientific).

Aliquots of 1×10⁶ cells were resuspended in 2.0 ml PBS containing 5.0 UM CFSE (Carboxyfluorescein diacetate succinimidyl ester, Molecular Probes, USA) for 10 minutes at 37° C. This reaction was stopped by adding 5 volumes of 10% RPMI (Gibco) and ice-cold FBS (Thermo Scientific), then the cell suspension was incubated for 5 minutes on ice in the dark.

After this procedure, the cells were washed in PBS 3 times, counted, and used for bioprinting.

The images were performed on a Leica TCS SP8 confocal laser scanning microscope.

The resazurin viability assay has been widely used since the intracellular reaction of converting the non-fluorescent oxidized form of resazurin into the reduced fluorescent form can be detected by spectrophotometry, identifying metabolically active cells (BONNIER et al., 2015).

For this assay, the biodressings were removed from the culture medium and incubated in a resazurin solution (Sigma-Aldrich) (0.025 mg/mL in PBS) for 6 hours at 37° C. in an incubator with 5% CO₂. After incubation, the fluorescence intensity was analyzed in a spectrophotometer (λ excit 540 nm and λ emi 590 nm).

Both MCUs grown under usual conditions (MCU FBS) and those grown free of xenoantigens (MCU AB) showed adequate cell viability after bioprinting. The kinetics of cell viability of both MCU FBS and MCU AB showed a brief increase around the third day of culture, followed by a decrease, but remained viable for the period analyzed.

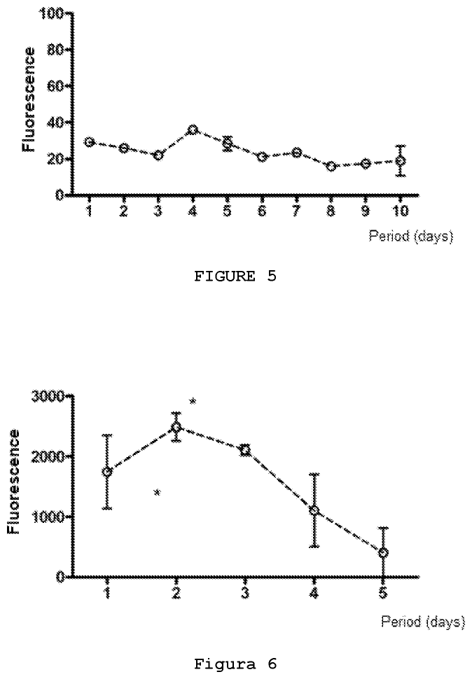

FIG. 5 graphically represents the kinetics of cell viability of bioprinted MCUs FBS, in which the kinetics are evaluated by staining with resazurin fluorescent dye, and the results are expressed concerning the average of at least three biodressings per period considered.

FIG. 6 graphically represents the kinetics of cell viability of bioprinted AB MCUs, in which the kinetics is evaluated by staining with resazurin fluorescent dye, and the results are expressed concerning the average of at least three biodressings per period evaluated.

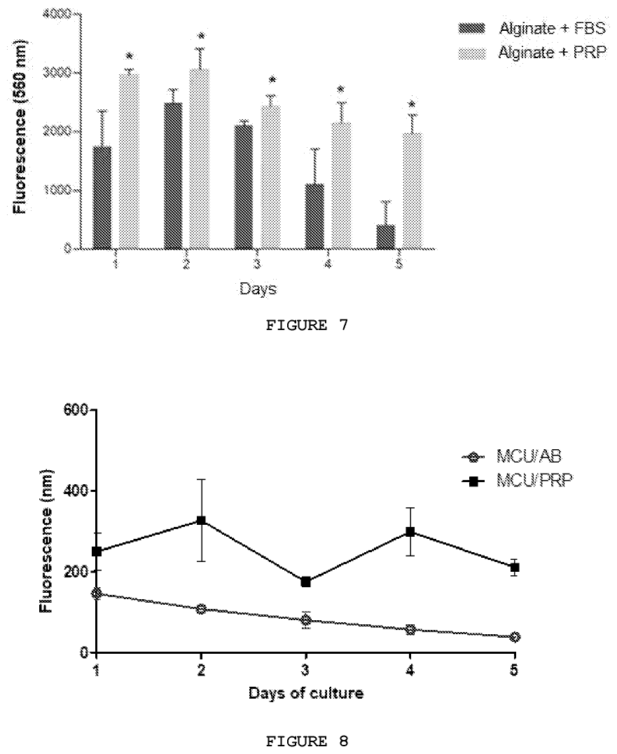

In order to improve the viability of bioprinted cells and obtain another alternative for cell culture free of xenoantigens, the cultivation of 3D biodressings in platelet-rich plasma (PRP) was also tested, as mentioned previously. PRP is a potent stimulant of in vitro cell growth (SILVA et al., 2018; STESSUK et al., 2016), and the addition of 10% of PRP replacing FBS after bioprinting the cells enhanced the cell growth in 3D biodressings from the first day of cultivation, lasting until the fifth day, as shown in FIG. 7.

FIG. 7 graphically represents the kinetics of cell viability of bioprinted MCUs in the presence of DMEM culture medium supplemented with 10% FBS and DMEM culture medium supplemented with 10% PRP, in which the kinetics is evaluated through the staining with resazurin fluorescent dye. The results are expressed concerning the average of at least three 3D biodressings per period evaluated.

The same behavior was observed when evaluating the kinetics of 3D biodressings containing MCUs cultured free of xenoantigens, i.e., cell culture supplemented with AB or PRP serum. As in the previous experiments, the addition of PRP to the culture medium of the 3D biodressings enhanced cell growth compared to AB serum.

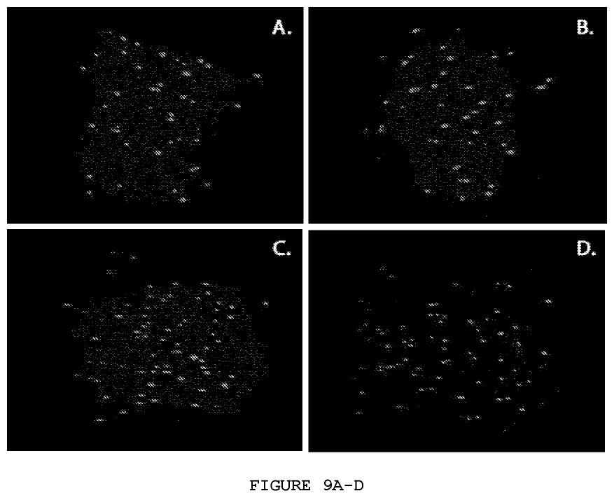

FIG. 8 graphically represents the comparison of the cell viability of MCUs in the biodressing in a culture medium with 10% PRP and 10% human AB serum. The biodressings of MCUs in alginate had their growth monitored for 5 days either when grown in culture medium with 10% human AB serum (MCU/AB) or 10% PRP (MCU/PRP). Cell viability is inferred by quantifying the fluorescence in nm emitted by the reaction with resazurin after 6 h of incubation.

Thus, since 3D cell culture offers an extra challenge to cell growth compared to two-dimensional monolayer culture, PRP represents a suitable cell growth stimulator.

The assessment of the cell viability of the AB MCUs contained in the 3D biodressings was confirmed by confocal microscopy (qualitative), with the MCUs remaining viable during the period analyzed (10 days after bioprinting) (FIG. 9).

### Healing Assay in Ex Vivo Human Skin Model and TGF-β and KGF Dosage:

The ex vivo healing assay in a human skin model was performed by Kosmoscience, according to the following protocol:

Fragments of human skin obtained from elective plastic surgery were subjected to tissue injury by scalpel and punch and treated with the biodressing of the present invention for 144 hours. Then, we performed the histological and quantification evaluation of growth factors TGF β and KGF.

### Human Skin Culture:

The skin fragments used in this study came from one (01) healthy individual, female, phototype III, 56 years old, who underwent elective plastic surgery in the abdomen (abdominoplasty). After the surgical procedure, the skin fragments were collected in plastic bottles containing 0.9% saline solution and kept refrigerated for up to 24 hours. This project did not include storing biological material for future use, and the spare fragments were properly discarded as infectious waste. The use of human skin fragments from elective surgeries for this study was submitted to the Research Ethics Committee of the Universidade Sao Francisco—SP, CAAE 82685618.9.0000.5514, under opinion 2.493.285.

### Treatment Protocol:

The skin fragments were fractionated into approximately 1.5 cm², placed in culture plates (Corning, USA) with an appropriate culture medium, and kept in an incubator at 37° C. with 5% CO₂. Then, they were submitted to tissue injury with a scalpel and punch and treated with the biodressing of the present invention for 144 hours. After this period, the fragments were submitted for histological analysis to evaluate the epidermal re-epithelialization and measurement of the transforming growth factor (TGF-β) and keratinocyte growth factor (KGF) by ELISA.

### TGF-β and KGF Quantification:

Quantifications of TGF-β and KGF mediators were performed in the supernatant by enzyme immunoassay, using kits purchased commercially (R&D, BD). The absorbance reading was performed at 450 nm in a Multiskan GO monochromator (Thermo Scientific).

### Statistical Analysis:

For the statistical analysis, the ANOVA test measured the variation of the results and compared the data between the groups. Then, the Bonferroni post-test was employed, reinforcing and making the result presented in the ANOVA test even more accurate. A significance level of 5% (GraphPad Prism v6) was used.

In the skin healing process, keratinocytes play a central role not only as a critical structural cell in regenerated skin but also as a source of growth factors and stimulation of cell proliferation and migration, such as keratinocyte growth factor (KGF), demonstrating a crucial role in tissue repair.

The production of inflammatory mediators, such as PGE2 and IL-6, associated with injured skin, as well as the intrinsic and extrinsic skin aging process, is an essential factor in the exacerbation of the immune response that can negatively interfere with the process of skin healing, disharmonizing the stages of re-epithelialization, cohesion, and epidermal hydration.

Thus, the availability of products with regenerating properties through the optimization of the activity of fibroblasts and the stimulus of hydration and skin cohesion, improving the production of the proteins described above, can be a differential in the tissue repair process.

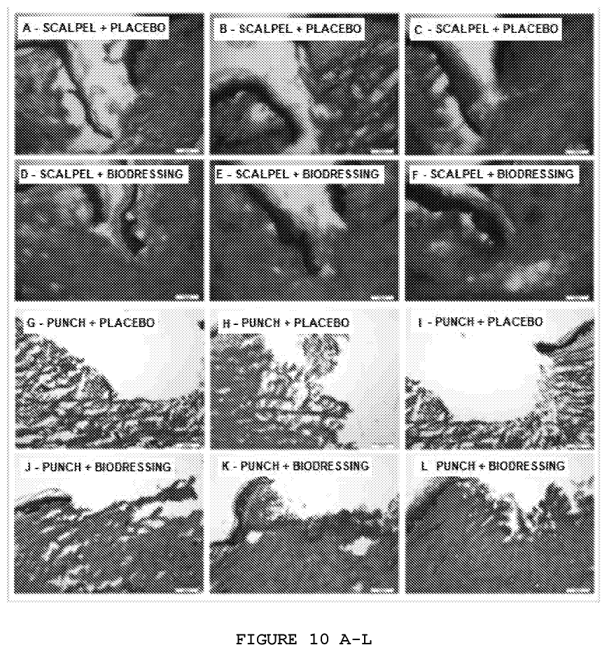

FIG. 10 shows the results of epidermal re-epithelialization from ex vivo skin fragments.

FIG. 10 demonstrates an apparent regeneration mainly in the dermal region, following the homeostatic sense of renewal—from the reticular dermis to the fiber the papillary. Furthermore, density after treatment with the 3D biodressing is greater compared the placebo group (3D Biodressing without MCUs).

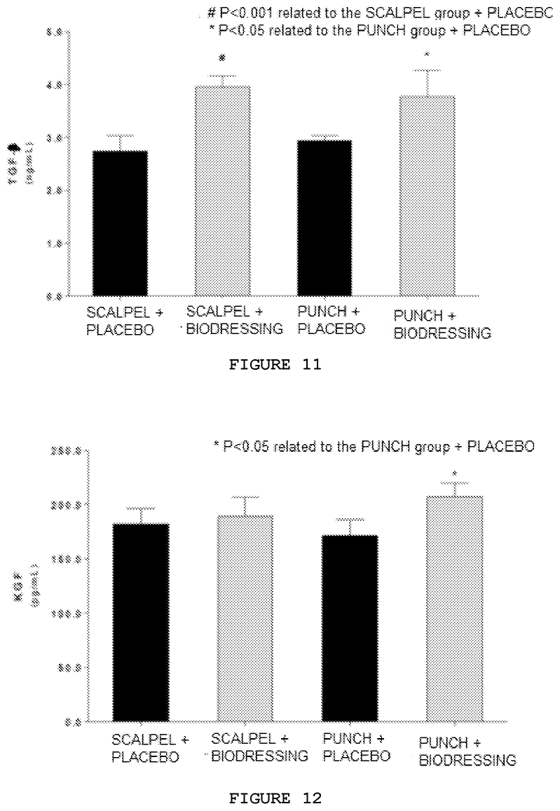

FIGS. 11 and 12 show the results of the TGF β and KGF quantification from ex vivo skin fragments subjected to tissue injury by scalpel and punch. In FIG. 11, the fragments subjected to scalpel injury followed by treatment with the 3D biodressing of the present invention produced a 39.74% increase in TGF-β production compared to the PLACEBO group (P<0.001). The fragments submitted to punch injury followed by treatment with the 3D biodressing of the present invention produced an increase of 28.31% in the production of TGF-β compared to the PLACEBO group (P<0.05).

In FIG. 12, the fragments subjected to scalpel injury followed by treatment with the 3D biodressing of the present invention produced no change in KGF production compared to the PLACEBO group. On the other hand, the fragments submitted to punch injury followed by treatment with the 3D biodressing of the present invention produced a 21.11% increase in KGF production compared to the PLACEBO group (P<0.05).

The results allow us to infer that the 3D biodressing of the present invention exerts a positive effect on the tissue repair and regeneration process, supporting the skin healing process by increasing the production of TGF-β and KGF.

### T Cell Proliferation Immunosuppression Assay:

An essential therapeutic effect of CMs is based on their immunomodulatory potential. This property is attractive for the treatment of complex skin lesions since a well-orchestrated inflammatory phase of healing is fundamental for the optimal course of the entire process. The process of immunomodulation by CMs is described in numerous immune system cells (GAO, 2016); in this sense, we aimed to answer whether the immunomodulatory potential of CMs would be maintained after the process of bioprinting and cultivation of these cells in biodressings.

For this, a lymphocyte proliferation inhibition assay was carried out in the presence of 3D biodressing, e concentrations of CMs and PBMCs (peripheral blood mononuclear cell) were tested, 1 CM for 1 PBMC (1:1), 1 CM for 2 PBMCs (1:2) and 1 CM for 4 PBMCs (1:4), thus since the concentration of CMs in the biodressings is stable, only the number of PBMCs varied.

The 3D biodressings containing CMs were co-cultured with peripheral blood mononuclear cells (PBMC) stained with Carboxyfluorescein Succinimidyl Ester (CFSE) at different concentrations and stimulated with Phytohemagglutinin (PHA). Lymphocyte proliferation was analyzed by CFSE dilution by flow cytometry. In flow cytometry, T lymphocytes were selected by staining them with anti-CD3, anti-CD-4, and anti-CD8 antibodies (BD, Bioscience).

In the flow cytometry, anti-CD3+anti-CD4 antibodies were added to identify CD4 T lymphocytes or anti-CD3+anti-CD8 to quantify CD8 T lymphocytes.

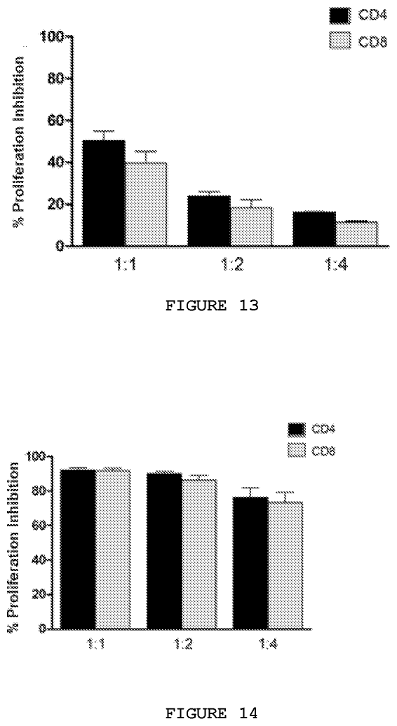

After 3 days of co-cultivation of the 3D biodressings with PBMCs, we observed a percentage of inhibition of lymphocyte proliferation of approximately 50% for the highest concentration of CMs used (1:1), about 30% for concentration 1:2, and 20% for the 1:4 concentration (FIG. 13), and the percentage of proliferation inhibition was similar for TCD4 lymphocytes and TCD8 lymphocytes.

FIG. 13 graphically represents the lymphocyte proliferation inhibition potential of 3D biodressings, in which PBMCs are stained with CFSE (5 μM) at different concentrations and stimulated with PHA (2 μg/mL). The results refer to the third day of co-cultivation, and to calculate the inhibition potential, the following formula was used: [(% CD3+CFSE low cells−CD3+CFSE low cells co-cultured with the biodressings) % CD3+CFSE low cells]×100. Results are expressed as mean±standard deviation.

After five days of co-cultivation of 3D biodressings with PBMCs, flow cytometry analyses showed that high rates of inhibition of lymphocyte proliferation were obtained, reaching about 90% for concentrations 1:1 and 1:2 and 80% for the 1:4 concentrations, both for TCD4 lymphocytes and TCD8 lymphocytes (FIG. 14).

FIG. 14 graphically represents the lymphocyte proliferation inhibition potential of 3D biodressings, in which PBMCs are stained with CFSE (5 UM) at different concentrations and stimulated with PHA (2 μg/mL). The results refer to the fifth day of co-culture, and to calculate the inhibition potential, the following formula was used: [(% CD3+CFSE low cells CD3+CFSE low cells co-cultured with the biodressings) % CD3+CFSE low cells]×100. The results are expressed as mean±standard deviation.

These results suggest that the immunomodulatory potential of the MSCs of the biodressings is maintained after their manufacture, indicating the potent anti-inflammatory effect of the 3D biodressings produced.

### Assay to Assess the Nociceptive Threshold:

Wounds are injuries that can be extremely painful, pain in most cases is often the main complaint of these patients. Reducing the pain of patients with chronic wounds or severe burns is relevant as it improves their quality of life.

In this sense, the analgesic potential of 3D biodressings was tested in a carrageenan-induced hyperalgesia model in rats.

Six Wistar rats (300-350 g) were used per experimental group.

Hyperalgesia was induced by applying 100 μg/50 μL/paw carrageenan solution. The carrageenan solution was made by diluting the drug in 0.9% NaCl (saline) immediately before use. The 100 μg dose has been shown to induce hyperalgesia, whose peak occurs 3 h after administration and persists for at least 24 h (Bonet et al., 2013).

Since much of the therapeutic effect of MSCs comes from their paracrine effect (Meirelles, 2009), samples of the culture supernatant of the 3D biodressings were collected 3 and 7 days after the biodressings cultivation. For this assay, animals submitted to the hyperalgesia model with carrageenan were treated with local application of the supernatant of the biodressing culture.

After the induction of hyperalgesia, 50 μL of the supernatant of the culture of the biodressing with MSCs collected 3 and 7 days after the impression of the biodressing were injected into the animals' paws. The control of the experiment was done through the injection of 50 μL of culture medium not conditioned. All drugs were injected into the subcutaneous tissue of the back of the hind paw.

Mechanical hyperalgesia was quantified using the Randall-Sellito test for paw removal (Randall & Sellito, 1957). This equipment has a compression device with two items: a flat surface on which the sole of the paw rests and a conical part that exerts pressure on the back of the paw. During the test, the experimenter presses a pedal that transmits to the conical portion a tension that increases linearly, pressing the rat's paw. This pressure is quantified by a movable cursor that slides over a scale and shows, in grams, the pressure exerted. When the rat withdraws its paw, the experimenter releases the pedal to stop the tension indicator and reads the scale. The Paw Withdrawal Threshold was defined as the average of three measurements taken alternately.

The animals were randomly divided into 3 experimental groups. On the day of the experiment, the treatments were evaluated as follows: the baseline paw withdrawal threshold and soon after, Carrageenan (100 μg/50 μL) was administered to the dorsum of the hind paw, and the baseline paw withdrawal threshold was evaluated at 1 h and 2 h after the injection. Immediately after the assessment at 2 h after the carrageenan injection (1 h before the peak of carrageenan-induced hyperalgesia), the rats received one of the following treatments on the dorsum of the ipsilateral hind paw to the one that received carrageenan:
Group 1: biodressing supernatant with mesenchymal cells collected 3 days after printing the biodressing;
Group 2: biodressing supernatant with mesenchymal cells collected 7 days after printing the biodressing;
Group 3: cell culture medium (vehicle). The Paw Withdrawal Threshold was assessed immediately, 1 h and 2 h after treatment.

A two-way analysis of variance (two-way ANOVA) was performed to analyze whether there were differences between the different groups throughout the experiment, followed by the Bonferroni test. One-way analysis was performed using a one-way analysis of variance (one-way ANOVA) followed by Tukey's test. The accepted level for statistical significance was p<0.05.

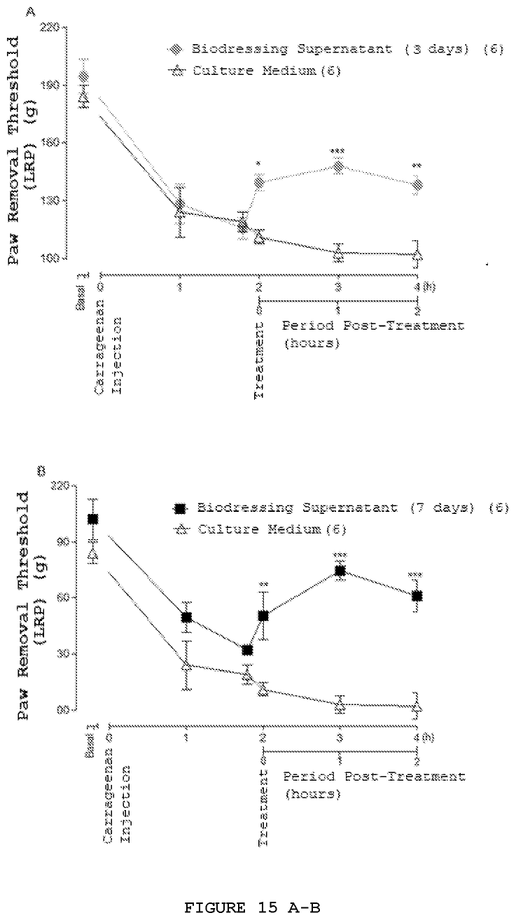

Animals treated with 3D biodressing supernatant collected 3 and 7 days after biodressing printing had a significantly higher Paw Withdrawal Threshold than those in the group that received only culture medium at 0, 1, and 2 hours after treatments (FIG. 15).

FIG. 15A-B graphically represents the effect of biodressing supernatant with MSCs collected 3 (A) and 7 (B) days after biodressing printing on carrageenan-induced hyperalgesia in rats. Carrageenan (100 μg/50 μL) was administered to the back of the hind paw of rats. After 2 h, the same paw received 50 μL of cell culture medium (vehicle) or biodressing supernatant with mesenchymal cells-3 days (A) or 7 days (B). Both treatments increased Paw Withdrawal Threshold immediately, 1 and 2 hours after injection (2-Way ANOVA for repeated measures followed by Bonferroni test. *p<0.05; **p<0.01; ***p<0.001 indicates significantly higher Paw Withdrawal Threshold (analgesia) than the group of animals that received the culture medium. Data are expressed as mean±standard error of the mean of 6 rats/group).

As the increase in the Paw Withdrawal Threshold represents analgesia, the results demonstrate that 3D biodressing supernatant with MSCs has an analgesic effect.

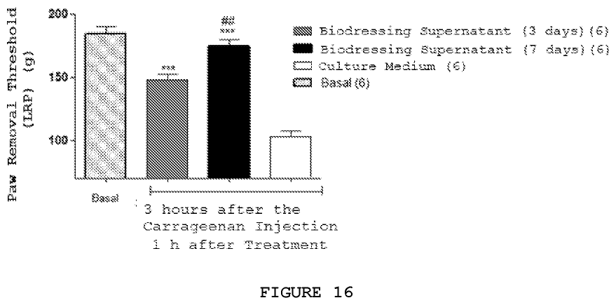

The peak of inflammatory hyperalgesia induced by carrageenan occurs 3 h after its administration (BONET, 2013). Rats were treated 2 hours after carrageenan injection, and Paw Withdrawal Threshold was assessed 0, 1, and 2 hours later. The effect of treatments on Paw Withdrawal Threshold 3 hours after carrageenan injection is shown in FIG. 16. Both groups treated with the biodressing supernatant collected 3 days after bioprinting and those treated with the 7-day biodressing had a significantly higher Paw Withdrawal Threshold than that of animals treated with culture medium (FIG. 16), demonstrating the analgesic effect of the treatments on the peak of carrageenan-induced hyperalgesia. The Paw Withdrawal Threshold of the group treated with 7-day biodressing supernatant was similar to baseline Paw Withdrawal Threshold and significantly higher than the Paw Withdrawal Threshold of the group treated with 3-day biodressing supernatant and culture medium, indicating that the 7-day biodressing supernatant has a more significant analgesic effect than the 3-day biodressing supernatant.

FIG. 16 represents the effect of MSCs biodressing supernatant collected 3 and 7 days after biodressing printing on carrageenan-induced hyperalgesia in rats. In the 3rd hour after the carrageenan injection and 1 h after treatments, rats treated with biodressing supernatant had a higher Paw Withdrawal Threshold than those receiving culture medium (indicated by “***”). Treatment with 7-day biodressing supernatant induced a more significant analgesic effect than 3-day biodressing supernatant (indicated by “##”). ***p<0.001 indicates a significantly higher Paw Withdrawal than the group that received the culture medium; ##p<0.01 indicates a significantly higher Paw Withdrawal Threshold than the group that received the biodressing supernatant collected 3 days after the biodressing printing. Data are expressed as mean±standard error of the mean of 6 rats/group.

Therefore, the development of 3D biodressings using adipose tissue-derived mesenchymal cells (ADSCs) and umbilical cord-derived cells (MCUs) is unprecedented and has been shown to be viable in in vitro experiments.

The possibility of using cells of allogeneic origin from pre-existing cell banks may represent the agility of patient treatment and scalability of production; furthermore, the use of less mature cells (MCUs) from more young individuals is promising in terms of therapeutic potential.

Although regulatory agencies currently do not require that cells intended for cell therapy be cultured under xenoantigen-free conditions, the process of obtaining the 3D biodressing of the present invention using mesenchymal cells proved to be viable, therefore, the product obtained is suitable for these characteristics, which provides more safety.

Finally, functional assays have shown that 3D biodressings containing CMs have an anti-inflammatory and analgesic effect; they also indicate that the healing potential of CMS is maintained after the bioprinting process.

Thus, the accomplishments presented in this invention do not limit the totality of possibilities. Various omissions, substitutions, and alterations can be made by a skilled person, keeping the spirit and scope of the present invention.

It is provided that all combinations of elements that perform the same role substantially in the same way to achieve the same results are within the scope of the invention. Element substitutions from one described experiment to another are also fully intended and contemplated.

It is also necessary to understand that the designs are not necessarily to scale but are only conceptual.

Those skilled in the subject will appreciate the knowledge presented herein and will be able to reproduce the invention in the proposed experiment and other variants covered by the scope of the claims.

### BIBLIOGRAPHIC REFERENCES

ADRIANO FARINA, J. et al. Autologous adipose-derived stem cell for painful leg ulcers in patients with sickle cell disease. A preliminary study. British Journal of Haematology, 2019.
ATIYEH, B. S.; GUNN, S. W.; HAYEK, S. N. State of the art in burn treatment. World journal of surgery, v. 29, n. 2, p. 131-48, February 2005. BADILLO, A. T. et al. Treatment of diabetic wounds with fetal murine mesenchymal stromal cells enhances wound closure. Cell and tissue research, v. 329, n. 2, p. 301-11, August 2007.
ATIYEH, B. S.; GUNN, S. W.; HAYEK, S. N. State of the art in burn treatment. World journal of surgery, v. 29, n. 2, p. 131-48, February 2005.
BADILLO, A. T. et al. Treatment of diabetic wounds with fetal murine mesenchymal stromal cells enhances wound closure. Cell and tissue research, v. 329, n. 2, p. 301-11, August 2007.
BARTHEL, R.; ABERDAM, D. Epidermal stem cells. Journal of the European Academy of Dermatology and Venereology: JEADV, v. 19, n. 4, p. 405-13, July 2005.
BELLINI, Z. et al. Combining xanthan and chitosan membranes to multipotent mesenchymal stromal cells as bioactive dressings for dermo-epidermal wounds. Journal of biomaterials applications, v. 0, n. 0, p. 1-12, 2014.
BOLAND, T. et al. Drop-on-demand printing of cells and materials for designer tissue constructs. Material Science & Engineering, v. 27, p. 372-376, 2007.
BONET, I J M; FISCHER, L; PARADA, C A; TAMBELI, CH. (2013) The role of Transient Receptor Potencial A 1 (TRPA1) in the development and maintenance of carrageenan-induced hyperalgesia. Neuropharmacology, 65:206-212.
BONNIER, F. et al. Cell viability assessment using the Alamar blue assay: A comparison of 2D and 3D cell culture models. Toxicology in Vitro, v. 29, p. 124-131, 2015. BOULTON, A. J. et al. The global burden of diabetic foot disease. Lancet, v. 366, n. 9498, p. 1719-1724, 2005.
CALIARI-OLIVEIRA, C. et al. Xenogeneic mesenchymal stromal cells improve wound and modulate the immune response in an extensive burn model. Cell Transplantation, 2015.
CLARK, M. Alginates in Dressings and Wound Management. Birmingham: [s.n.].
COLOMBO, M.; RAPOSO, G.; THERY, C. Biogenesis, Secretion, and Intercellular Interactions of Exosomes and Other Extracellular Vesicles. Annual Review of Cell and Developmental Biology, v. 30, n. 1, p. 255-289, 2014.
COSTA, A. M. et al. Custos do tratamento de hlceras por pressao em unidade de cuidados prolongados em uma instituigao hospitalar de Minas Gerais. Enfermagem revista, v. 18, p. 58-74, 2015.
DAI, W. L. et al. Efficacy of Platelet-Rich Plasma in the Treatment of Knee Osteoarthritis: A Meta analysis of Randomized Controlled Trials. Arthroscopy Journal of Arthroscopic and Related Surgery, v. 33, n. 3, p. 659-670.el, 2017.
DE-SOUZA, D.; MARCHESAN, W. G.; GREENE, L. J. Epidemiological data and mortality rate of patients hospitalized with burns in Brazil. Burns, v. 24, p. 433-438, 1998.
DELANEY, K.-M. H. et al. Leg Ulcers in Sickle Cell Disease: Current Patterns and Practices. Hemoglobin, v. 37, n. 4, p. 325-332, 2013.
DELGADO, J. C.; LA, E. Preface H L A in Tr a nsplantation a n d Beyond. Clin and Lab Med, v. 38, 2018.
DOMINICI, M. et al. Minimal criteria for defining multipotent mesenchymal stromal cells. The International Society for Cellular Therapy position statement. Cytotherapy, v. 8, n. 4, p. 315-7, January 2006.
E L OMAR, R. et al. Umbilical Cord Mesenchymal Stem Cells: The New Gold Standard for Mesenchymal Stem Cell-Based Therapies? Tissue Engineering Part B: Reviews, v. 20, n. 5, p. 523-544, 2014.
ENOCH, S.; GREY, J. E.; HARDING, K. G. Recent advances and emerging treatments. BMJ (Clinical research ed.), v. 332, n. 7547, p. 962-5, 22 Apr. 2006.
FALANGA, V. Wound healing and its impairment in the diabetic foot. Lancet, v. 366, p. 1736-1743, 2005.
FALANGA, V. et al. Autologous bone marrow-derived cultured mesenchymal stem cells delivered in a fibrin spray accelerate healing in murine and human cutaneous wounds. Tissue engineering, v. 13, n. 6, p. 1299-312, June 2007.
FOLEY, E.; ROBINSON, A.; MALONEY, M. Skin Substitutes and Dermatology: A Review. Current Dermatology Reports, v. 2, n. 2, p. 101-112, 2 Apr. 2013.
FONDER, M. A. et al. Treating the chronic wound: A practical approach to the care of nonhealing wounds and wound care dressings. Journal of the American Academy of Dermatology, v. 58, p. 185-206, 2008.
Gao, F; Chiu, S M; Motan, D A L; Zhang, Z; Chen, L; Ji, H-L; Tse, H-F; Fu, Q-L; Lian, Q. Mesenchymal stem cells and immunomodulation: current status and future prospects. Cell Death and Disease. 7, 1, 2016.
GAO, G. et al. Bioactive nanoparticles stimulate bone tissue formation in bioprinted three-dimensional scaffold and human mesenchymal stem cells. Biotechnology Journal, p. 1-23, 2014.
GAO, G. et al. Improved properties of bone and cartilage tissue from 3D inkjet-bioprinted human mesenchymal stem cells by simultaneous deposition and photocrosslinking in PEG-GelMA. Biotechnology Letters, v. 37, n. 11, p. 2349-2355, 2015.
GROEBER, F. et al. Skin tissue engineering in vivo and in vitro applications. Advanced drug delivery reviews, v. 63, n. 4-5, p. 352-66, 30 Apr. 2011.
HALABI-TAWIL, M. et al. Sickle cell leg ulcers: A frequently disabling complication and a marker of severity. British Journal of Dermatology, v. 158, n. 2, p. 339-344, 2008.
HASS, R. et al. Different populations and sources of human mesenchymal stem cells (MSC): A comparison of adult and neonatal tissue-derived MSC. Cell Communication and Signaling, v. 9, p. 1-14, 2011.
HE, Y. et al. Research on the printability of hydrogels in 3D bioprinting. Nature Publishing Group, n. 6, p. 1-13, 2016. HOFFMANN, M. et al. Spatial organization of mesenchymal stem cells in vitro—results from a new individual cell-based model with podia. Plos one, v. 6, n. 7, p. e21960, January 2011.
HORWITZ, E. et al. Clarification of the nomenclature for MSC. Cytotherapy, v. 7, n. 5, p. 393-395, 2005.
JAVAZON, E. H. et al. Enhanced epithelial gap closure and increased angiogenesis in wounds of diabetic mice treated with adult murine bone marrow stromal progenitor cells. Wound repair and regeneration: official publication of the Wound Healing Society [and] the European Tissue Repair Society, v. 15, n. 3, p. 350-9, 2007.
JI, S.; GUVENDIREN, M. Recent Advances in Bioink Design for 3D Bioprinting of Tissues and Organs. Frontiers in Bioengineering and Biotechnology, v. 5, n. April, p. 1-8, 2017.
KATAOKA, K.; MEDINA, R. J. Short Communication Participation of Adult Mouse Bone Marrow Cells in Reconstitution of Skin. American journal of pathology, v. 163, n. 4, p. 1227-1231, 2003.
KOGA, A. Y. et al. Evaluation of wound healing effect of alginate films containin g Aloe vera (Aloe barbadensis Miller) gel. Biomaterials applications, p. 1-10, 2018.
LACCI, K. M.; DARDIK, A. Platelet-Rich Plasma: Support for Its Use in Wound Healing. Yale Journal of Biological and medicien, v. 83, p. 1-9, 2010. LAI, R. C.; YEO, R. W. Y.; LIM, S. K. Mesenchymal stem cell exosomes. Seminars in Cell and Developmental Biology, v. 40, p. 82-88, 2015.
LE BLANC, K. et al. HLA expression and immunologic properties of differentiated and undifferentiated mesenchymal stem cells. Experimental Hematology, v. 31, p. 890-896, 2003.
MAVER, T. Polysaccharide Based Wound Care. [s.1: s.n.].
MCCAVIT, T. L. Sickle Cell Disease. Pediatrics in Review, v. 33, n. 5, p. 195-206, 2012.
MEIRELLES, L. D. S. et al. Mechanisms involved in the therapeutic properties of mesenchymal stem cells. Cytokine & growth factor reviews, v. 20, n. 5-6, p. 419-27, 2009.
MEIRELLES, S.; CHAGASTELLES, P. C.; NARDI, N. B. Mesenchymal stem cells reside in virtually all post-natal organs and tissues. Journal of cell science, v. 119, n. 11, p. 2204-2213, 2006.
MINNITI, C. P. et al. Leg ulcers in sickle cell disease. American Journal of Hematology, v. 85, n. 10, p. 831-833, 2010.
MIR, M. et al. Synthetic polymeric biomaterials for wound healing: a review. Progress in Biomaterials, 2018.
PIEL, F. B. et al. Global Burden of Sickle Cell Anaemia in Children under Five, 2010-2050: Modelling Based on Demographics, Excess Mortality, and Interventions.
PLOS Medicine, v. 10, n. 7, 2013. RANDALL, LO & SELLITO, J J. A method for measurement of analgesic activity on inflamed tissue. Arch nt Pharmacod n therapie 111:409-419, 1957.
RANI, S. et al. Mesenchymal stem cell-derived extracellular vesicles: Toward cell-free therapeutic applications. Molecular Therapy, v. 23, n. 5, p. 812-823, 2015.
RATNER, B. D. et al. Biomaterials science, an introduction to materials in medicine. San Diego: Spciety
RATNER, B. D.; BRYANT, S. J. Biomaterials: where we have been and where we are going. Annual review of biomedical engineering, v. 6, n. 1, p. 41-75, January 2004.
SCHREML, S.; BERNEBURG, M. The global burden of diabetic wounds. British Journal of Dermatology, v. 176, n. 4, p. 845-846, 2017.
SEN, C. K. et al. Human skin wounds: A major and snowballing threat to public health and the economy: PERSPECTIVE ARTICLE. Wound Repair and Regeneration, v. 17, p. 763-771, 2009.
SERJEANT, G. Leg ulceration in Sickle Cell Anaemia. Archives of Internal Medicine, v. 133, n. 4, p. 690-694, 1974.
SHORES, J. T., GUPTA, G. A., Skin substitutes and alternatives: a review. Advances in skin & wound care. 20, 9, 494-508, 2007.
SILVA, A. et al. Platelet-rich plasma lyophilization enables growth factor preservation and functionality when compared with fresh platelet-rich plasma. Regenerative Medicine, 2018.
SKARDAL, A. et al. Bioprinted Amniotic Fluid-Derived Stem Cells Accelerate Healing of Large Skin Wounds. Stem Cells Translational Medicine, v. 1, p. 70-78, 2012.
STESSUK, T. et al. Platelet-rich plasma (PRP) and adipose-derived mesenchymal stem cells: stimulatory effects on proliferation and migration of fibroblasts and keratinocytes in vitro. Archives of dermatological research, p. 1-10, 2016.
UCCELLI, A.; MORETTA, L.; PISTOIA, V. Immunoregulatory function of mesenchymal stem cells. European journal of immunology, v. 36, n. 10, p. 2566-73, October 2006.
VITTAL, B. A Growing Market: Wound Care Management-Hospital Management. Management, n. October 2010, p. 3-6, 2010.
VOLK, S. W. et al. Stromal progenitor cell therapy corrects the wound-healing defect in the ischemic rabbit ear model of chronic wound repair. Wound repair and regeneration: official publication of the Wound Healing Society [and] the European Tissue Repair Society, v. 15, n. 5, p. 736-47, 2007.
WU, Y. et al. Mesenchymal stem cells enhance wound healing through differentiation and angiogenesis. Stem cells (Dayton, Ohio), v. 25, n. 10, p. 2648-59, October 2007.
ZHANG, P. et al. Global epidemiology of diabetic foot ulceration: a systematic review and meta-analysist. Annals of Medicine, v. 49, n. 2, p. 106-116, 2017.

1.-25. (canceled)

26. A process to obtain a three-dimensional (3D) biodressing, comprising:
three-dimensionally bioprinting mesenchymal cells cultured in a xenoantigen-free medium in an alginate hydrogel to create the 3D biodressing;
resting the 3D biodressing for at least 5 minutes after the bioprinting; and
crosslinking the 3D biodressing in a calcium chloride solution after the resting.

27. The process according to claim 26, wherein the mesenchymal cells are selected from mesenchymal cells derived from human adipose tissue (ADSCs) and mesenchymal cells derived from the human umbilical cord (MCUs).

28. The process according to claim 26, wherein the mesenchymal cells are cultured in human AB serum.

29. The process according to claim 26, wherein the bioprinting comprises a layer-by-layer deposition of a mesh, with 2 to 10 layers, and wherein the cells are arranged within the alginate hydrogel.

30. The process according to claim 26, wherein the alginate hydrogel is a 4% w/v sodium alginate hydrogel.

31. The process according to claim 26, wherein the bioprinting is performed at a concentration of mesenchymal cells of 0.5×10⁵ to 1×10⁶ cells/mL.

32. A three-dimensional (3D) biodressing obtained by the process as defined in claim 26.

33. A process, comprising:
using the 3D biodressing obtained by the process as defined in claim 26 to treat a wound or a burn.

34. The process according to claim 33, wherein the using comprises applying the 3D biodressing to the wound or the burn.

35. The process according to claim 33, wherein the 3D biodressing has a healing, anti-inflammatory, and analgesic effect.

36. The process according to claim 33, wherein the 3D biodressing accelerates and improves the healing process and increases the production of TGF-b and KGF growth factors.
